(19) United States
(12) Patent Application Publication
(10) Pub. No.: US 2023/0021957 A1
(43) Pub. Date: Jan. 26, 2023

(54) USE OF PSILOCIN, PSILOCYBIN OR ANALOGS THEREOF IN WEIGHT LOSS, TREATMENT OF OBESITY AND COMPULSIVE EATING DISORDER

(71) Applicant: NEONMIND BIOSCIENCES INC., Vancouver (CA)

(72) Inventor: Penny White, Vancouver (CA)

(21) Appl. No.: 17/782,582

(22) PCT Filed: Dec. 3, 2020
(86) PCT No.: PCT/CA2020/051659

§ 371 Date: Jun. 3, 2022

Related U.S. Application Data
(60) Provisional application No. 62/943,337, filed on Dec. 4, 2019.

(51) Int. Cl. A61K 31/675; A61K 31/4045; A61K 36/07; A61P 3/04

(52) U.S. Cl. CPC A61K 31/675; A61K 31/4045; A61K 36/07; A61P 3/04

(57) ABSTRACT

Disclosed is a method of aiding in weight loss, treating compulsive eating disorder, treating obesity or a complication thereof, and/or altering the diet of an individual comprising administering psilocin, psilocybin, and/or an analog thereof.

### FIELD OF INVENTION

The invention relates to methods of using serotonin agonists, in particular, psychedelic mushroom actives, for weight loss.

### BACKGROUND

Obesity has been formally recognized by the World Health Organization as a global epidemic. Over 35% of adults in the United States, and more than 500 million adults on the planet are categorized as obese. Obesity is expected to cause or contribute to hundreds of thousands of deaths a year in the United States alone. Obesity reduces life expectancy, increases susceptibility to other disease states such as cardiovascular disease, high blood pressure, diabetes mellitus, hypertension, multiple sclerosis, erectile dysfunction, urinary incontinence, chronic renal failure, sleep apnea, asthma, and many forms of cancer. In addition to the clinically defined obese (those with a Body Mass Index (in kg/m²) of greater than 30), a large percentage of the population, while not obese, are overweight, which is undesired in many cultures. Obesity, and overweightness, is a leading preventable cause of death worldwide.

In most cases, obesity and overweightness is caused by a food energy intake that exceeds energy use. This is typically due to a combination of excessive eating and a lack of physical activity, though insufficient sleep, endocrine disruptors, a natural genetic disposition, and various medication use may also contribute. In almost all cases, obesity and overweightness can be treated by either or a combination of decreased caloric intake and increased exercise.

Unfortunately, decreasing caloric intake can be a very difficult task for many people. The Dieting industry is enormous, with some estimates showing that Americans spend over $60 billion annually on diet and weight loss products. Unfortunately, most of these diets do not work; much of this is thought to be because it is not enough to change food intake, an individual's behavior needs to change for long term success.

Many diet disorders exist, including binge eating disorder, also called compulsive eating disorder, where an individual will eat even when uncomfortably full or not hungry, often eating much faster than normal with a loss of control over how much or what is eaten. Compulsive overeating may also be ironically caused by a restrictive diet. Many people also overeat simply because they are hungry. Quality of diet also has a significant impact on obesity and overweightness, with low caloric content foods such as green vegetables contributing less to obesity than calorie dense foods which have high calories for their volume, such as refined carbohydrates. Interestingly, human food preferences (such as preferences for certain types of food) have been linked with a 5-HT2A serotonergic receptor polymorphism (Prado-Lima et al, Mol Psychiatry 2006 October). 5-HT2A receptors have also been associated with anorexia nervosa and bulimia nervosa. Obesity has been found, in some cases, to have associations with mood disorders (Simon et al., Archives of general psychiatry, 63(7), 824-830 (2006); Scott et al., Journal of psychosomatic research, 64(1), 97-105 (2008), DeWit et al., Psychiatry research, 178(2), 230-235 (2010)) and possesses neurobiological overlaps with certain classic forms of addiction (Volkow et al., Obesity reviews, 14(1), 2-18 (2013); Volkow et al., Biological psychiatry, 73(9), 811-818 (2013).

Obesity, over-eating, and an individual's relationship with food (including food choices) is an extremely complex, multi-factorial problem, which includes psychological issues, biochemical issues, as well as other issues such as social, cultural, and class issues. There is some evidence that the mesolimbic dopamine reward system is heavily implicated in the addictive role of both food and drugs of abuse (e.g. alcohol and nicotine). There has also recently been a line of evidence that suggests that specific kinds of foods that are high in sugars and fats may also induce behaviours that are hallmarks of addiction. For example, it has been suggested that rats will drink sugar-laced water in a binge-like manner, which will also subsequently release dopamine in the nucleus accumbens (Avena et al., Neuroscience, 139(3), 813-820 (2006); Rada et al., Neuroscience, 134(3), 737-744 (2005)). Thus, it has been suggested that obesity and disorders of addiction may have a similar neurobiological aetiology. Also, in healthy individuals with a BMI in the ‘normal’ range, there is similar functional activity associated with the consumption of foodstuff high in sugar and fats. Certain foods stimulate release of dopamine in the ventral striatum at a rate which is proportional to the rating of the pleasantness of the food. Food cravings have been found to be a predictor of treatment outcomes in relation to obesity; thus reduction of food cravings may be a useful focus in treatment of obesity and obesity-caused diseases/disorders or diseases/disorders to which obesity is a contributing factor.

Drugs that modify the serotonergic (5-hydroxytryptophan [5-HTP]) system have historically represented the most common and effective compounds for weight loss (Halford J C et al., Drugs 2007; 67(1):27-55). These include compounds such as sibutramine and the combination of fenfluramine/phentermine (known as “fen-phen”). Serotonin plays a key role in meal satiety, as increased serotonin levels in the brain result in decreased appetite, with subsequent weight loss. This is posited to occur through selective stimulation of the 5-HT1B, 5-HT2A and 5-HT2C serotonin receptors (Voigt J P and H. Fink, Behav Brain Res. 2015; 277:14-31; Currie P J and D V Coscina, Brain Res. 1998; 803(1-2):212-217). However, drugs such as fenfluramine/phentermine work by causing a general increase in serotonin levels. As there are at least 14 different types of serotonin receptors (Green, Br. J. Pharmacol. 2006; 147 Suppl 1 (Suppl 1): S145-52), this results in many “off target” serotonin receptors being stimulated, which in turn causes a host of unwanted side-effects (such as cardiac valvulopathies with fenfluramine/phentermine).

Psilocin, or 4-hydroxy-N,N-dimethyltryptamine, and its phosphorylated prodrug, psilocybin, were first isolated from Psilocybe mexicana in 1958. They have both since been synthetically produced. Structural analogs of psilocin, many having higher stability, are also known, such as 4-SH-dimethyltryptamine, 1-methylpsilocin, 4-fluoro-N,N-dimethyltryptamine, O-acetylpsilocin, 4-hydroxy-N-methyl-N-isopropyltryptamine, and 4-hydroxy-N-methyl-N-ethyltryptamine.

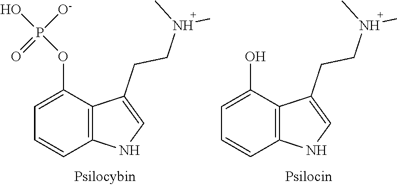 [SMILES: CN(C)CCc1c[nH]c2cccc(OP(=O)([O-])O)c12 ; CN(C)CCc1c[nH]c2cccc(O)c12]

Psilocin/psilocybin and its analogs are most commonly used as a psychoactive recreational drug, with a half-life of 1-3 hours when orally administered. Interestingly, they are known as 5-HT_{2A}, 5-HT_{2C} and 5-HT_{1A} agonists or partial agonists. Psilocybin is present in most psychedelic mushrooms, and at “normal” dosages creates mind-altering effects including euphoria, visual and mental hallucinations, changes in perception, a distorted sense of time, spiritual experiences, nausea, and panic attacks.

Psilocin has no approved or common medical use. However, so-called “microdoses” of psilocin (typically a fraction of the hallucination-inducing dosages) have been anecdotally reported to improve attention, and there have been numerous studies performed on its potential use for treating drug dependence, anxiety, mood disorders, and depression, with varying and largely inconclusive results to date. For example, the Heffter Research Institute (heffter.org) has published research indicating that psilocybin can relieve the symptoms of anxiety and depression often found in patients with a cancer diagnosis, as well as for treatment of addiction and obsessive-compulsive disorder. Usona Institute is currently in the planning phase for a phase 2/3 clinical research program on use of psilocybin as a treatment for major depressive disorder (www.usonaclinicaltrials.org). Trials have been recently undertaken have investigated the effects of psilocybin-assisted psychotherapy on major depression, including Carhart-Harris et al (2016), and Davis et al (2020). Interestingly, psilocybin assisted psychotherapy showed some efficacy in treating depression, notably, in cases where the depression was unresponsive to multiple types of conventional medication and psychotherapy. Other groups such as Bogenschutz et al (2015) have conducted proof-of concept studies to investigate the use of psilocybin for treatment of alcoholism, with promising results. Another pilot study, Johnson et al (2014) investigated the use of psilocybin as a psychotherapeutic tool to induce tobacco smoking cessation. This small trial suggested psilocybin may be effective for this use. There has been some evidence that it may induce neuroplastic change, including facilitating the extinction of fear memory and fear response (Zhang et al., (2013) Neuropharmacology 64: 403-413).

Psilocybin/psilocin, is a direct agonist at select serotonin receptors.

Psilocin and psilocybin are also used recreationally, both in small amounts (microdosing) and in larger amounts that induce hallucinatory effects. Microdose Nature (B C, Canada, www.microdosenature.com, the entirety of the site incorporated herein by reference) teaches two typical microdosing protocols: that of Dr. James Fadiman, which teaches 50-100 mg of Psilocybin cubensis fruiting bodies every third day, ideally before noon; and that of Paul Stamets of 50-100 mg/day for five days, followed by two days off. A microdose is typically between 10 to 400 mg of fruiting bodies per day (or an equivalent amount of purified or synthetically produced psilocin or psilocybin), with amounts higher than 400 mg per day considered “creative”, “recreational”, or hallucinatory dosages. Doses of over 5 g are generally not recommended.

Various sources of mushroom may be used; each has different potencies. One study has noted the following concentrations of psilocybin in mushrooms: Psilocybe Azurescens: 1.78%; Psilocybe Bohemica: 1.34%; Psilocybe Semilianceata: 0.98%; Psilocybe Baeocystis 0.85%; Psilocybe Cyanescens: 0.85%; Psilocybe Tampanensis: 0.68%; Psilocybe Cubensis: 0.63%; Psilocybe Weilii: 0.61%, all wt/wt. There are 120 different species of mushroom within the Psilocybe genus; other genera which may contain psilocybin include Conocybe, Copelandia, Galerina, Gymnopilus, Inocybe, Mycena, Panaeolus, Pholiotina, and Pluteus.

### SUMMARY OF THE INVENTION

According to one aspect of the present invention is provided a method of treating compulsive eating disorder in an individual in need thereof, comprising administering an effective amount of a compound comprising psilocin, psilocybin, or an analog thereof.

According to a further aspect of the present invention is provided a method of aiding in or causing weight loss in an individual in need thereof, comprising administering an effective amount of a compound comprising psilocin, psilocybin, or an analog thereof.

According to yet a further aspect of the present invention is provided a method of treating obesity in an individual in need thereof, comprising administering an effective amount of a compound comprising psilocin, psilocybin, or an analog thereof.

According to yet a further aspect of the present invention is provided a method of treating a complication associated with obesity in an individual in need thereof, comprising administering an effective amount of a compound comprising psilocin, psilocybin, or an analog thereof.

According to yet a further aspect of the present invention is provided a method of altering a diet of an individual in need thereof, comprising administering an effective amount of a compound comprising psilocin, psilocybin, or an analog thereof.

According to yet a further aspect of the present invention is provided a method of reducing food cravings in an individual in need thereof, comprising administering an effective amount of a compound comprising psilocin, psilocybin, or an analog thereof.

According to yet a further aspect of the present invention is provided a method of improving quality of diet in an individual in need thereof, comprising administering an effective amount of a compound comprising psilocin, psilocybin, or an analog thereof.

According to yet a further aspect of the present invention is provided a method of increasing metabolism in an individual in need thereof, comprising administering an effective amount of a compound comprising psilocin, psilocybin, or an analog thereof.

According to yet a further aspect of the present invention is provided a method of aiding a healthy individual to maintain a healthy weight, comprising administering an effective amount of a compound comprising psilocin, psilocybin, or an analog thereof.

According to yet a further aspect of the present invention is provided a method of decreasing food intake in an individual in need or desire thereof, comprising administering an effective amount of a compound comprising psilocin, psilocybin, or an analog thereof.

According to certain embodiments, the compound is contained within a dried mushroom powder of the genus Psilocybe.

According to certain embodiments, the compound is a purified psilocin or psilocybin in a pharmaceutically acceptable carrier.

According to certain embodiments, the purified psilocin or psilocybin is manufactured synthetically.

According to certain embodiments, the effective amount is 0.6 to 2.5 mg of psilocin per day.

According to certain embodiments, the effective amount is 50 to 500 mg of dried mushroom powder per day.

According to certain embodiments, the complication associated with obesity is sleep apnea, abnormal regulation of blood glucose, diabetes mellitus, cardiovascular disease, high blood pressure, hypertension, multiple sclerosis, erectile dysfunction, urinary incontinence, chronic renal failure, asthma, and/or cancer.

According to certain embodiments, the administering is an oral administration.

According to certain embodiments, the analog thereof is 4-SH-dimethyltryptamine, 1-methylpsilocin, 4-fluoro-N,N-dimethyltryptamine, O-acetylpsilocin, 4-hydroxy-N-methyl-N-isopropyltryptamine, or 4-hydroxy-N-methyl-N-ethyltryptamine.

According to a further aspect of the present invention is provided the use of psilocin, psilocybin, or an analog thereof, in the manufacturing of a medicament for the treatment of compulsive eating disorder.

According to a further aspect of the present invention is provided the use of psilocin, psilocybin, or an analog thereof, in the manufacturing of a medicament for weight loss.

According to a further aspect of the present invention is provided the use of psilocin, psilocybin, or an analog thereof, in the manufacturing of a medicament for the treatment of obesity or a complication associated with obesity.

According to a further aspect of the present invention is provided the use of psilocin, psilocybin, or an analog thereof, in the manufacturing of a medicament for the altering of a diet of an individual.

According to a further aspect of the present invention is provided the use of psilocin, psilocybin, or an analog thereof, in the manufacturing of a medicament for reducing food cravings, improving quality of diet, increasing metabolism, or aiding a healthy individual to maintain a healthy weight.

According to a further aspect of the present invention is provided the use of psilocin, psilocybin, or an analog thereof, in the manufacturing of a medicament for decreasing food intake in an individual in need or desire thereof.

According to a further aspect of the present invention is provided the use of psilocin, psilocybin, or an analog thereof for the treatment of compulsive eating disorder.

According to a further aspect of the present invention is provided the use of psilocin, psilocybin, or an analog thereof for weight loss.

According to a further aspect of the present invention is provided the use of psilocin, psilocybin, or an analog thereof for the treatment of obesity or a complication associated with obesity.

According to a further aspect of the present invention is provided the use of psilocin, psilocybin, or an analog thereof, for the altering of a diet of an individual.

According to a further aspect of the present invention is provided the use of psilocin, psilocybin, or an analog thereof, for reducing food cravings, improving quality of diet, increasing metabolism, or aiding a healthy individual to maintain a healthy weight.

According to a further aspect of the present invention is provided the use of psilocin, psilocybin, or an analog thereof, for decreasing food intake in an individual in need or desire thereof.

According to certain embodiments the psilocin, psilocybin, or analog thereof is contained within a dried mushroom powder of the genus Psilocybe.

According to certain embodiments, the psilocin, psilocybin, or analog thereof is a purified psilocin or psilocybin in a pharmaceutically acceptable carrier.

According to certain embodiments, the purified psilocin or psilocybin is manufactured synthetically.

According to certain embodiments, the psilocin, psilocybin, or analog thereof, is for administration in a dose of 0.6 to 2.5 mg of per day.

According to certain embodiments, the dose is 50 to 500 mg of dried mushroom powder per day.

According to certain embodiments, the complication associated with obesity is selected from the group consisting of sleep apnea, abnormal regulation of blood glucose, diabetes mellitus, cardiovascular disease, high blood pressure, hypertension, multiple sclerosis, erectile dysfunction, urinary incontinence, chronic renal failure, asthma, and cancer.

According to certain embodiments, the psilocin, psilocybin, or analog thereof is for oral administration.

According to certain embodiments, the analog thereof is 4-SH-dimethyltryptamine, 1-methylpsilocin, 4-fluoro-N,N-dimethyltryptamine, O-acetylpsilocin, 4-hydroxy-N-methyl-N-isopropyltryptamine, or 4-hydroxy-N-methyl-N-ethyltryptamine.

According to a further aspect of the present invention is provided psilocin, psilocybin, or an analog thereof for use in the treatment of compulsive eating disorder.

According to a further aspect of the present invention is provided psilocin, psilocybin, or an analog thereof for use in weight loss.

According to a further aspect of the present invention is provided psilocin, psilocybin, or an analog thereof for use in the treatment of obesity or a complication associated with obesity.

According to a further aspect of the present invention is provided psilocin, psilocybin, or an analog thereof, for use in the altering of a diet of an individual.

According to a further aspect of the present invention is provided psilocin, psilocybin, or an analog thereof, for use in reducing food cravings, improving quality of diet, increasing metabolism, or aiding a healthy individual to maintain a healthy weight.

According to a further aspect of the present invention is provided psilocin, psilocybin, or an analog thereof, for use in decreasing food intake in an individual in need or desire thereof.

According to certain embodiments, the psilocin, psilocybin, or analog thereof is contained within a dried mushroom powder of the genus Psilocybe.

According to certain embodiments, the psilocin, psilocybin, or analog thereof is in the form of a purified psilocin or psilocybin in a pharmaceutically acceptable carrier.

According to certain embodiments, the purified psilocin or psilocybin is manufactured synthetically.

According to certain embodiments, the psilocin, psilocybin, or analog thereof is for administration in a dose of 0.6 to 2.5 mg of per day.

According to certain embodiments, the dose is 50 to 500 mg of dried mushroom powder per day.

According to certain embodiments, the complication associated with obesity is selected from the group consisting of sleep apnea, abnormal regulation of blood glucose, diabetes mellitus, cardiovascular disease, high blood pressure, hypertension, multiple sclerosis, erectile dysfunction, urinary incontinence, chronic renal failure, asthma, and cancer.

According to certain embodiments, the psilocin, psilocybin, or analog thereof is for oral administration.

According to certain embodiments, the analog thereof is 4-SH-dimethyltryptamine, 1-methylpsilocin, 4-fluoro-N,N-dimethyltryptamine, O-acetylpsilocin, 4-hydroxy-N-methyl-N-isopropyltryptamine, or 4-hydroxy-N-methyl-N-ethyltryptamine.

### BRIEF DESCRIPTION OF FIGURES

The present invention will be further understood from the following description with reference to the Figures, in which:

FIG. 1 shows cumulative weight gain per day in untreated and psilocybin-administered rats.

FIG. 2 shows total weight gain over a 5 day period in untreated and psilocybin-administered rats.

FIG. 3 shows cumulative weight gain, as a percentage of body weight, in untreated and psilocybin administered rats.

FIG. 4 shows total weight gain over 5 days, as a percentage of body weight, in untreated and psilocybin administered rats.

### DETAILED DESCRIPTION

Unless otherwise explained, all technical and scientific terms used herein have the same meaning as commonly understood by one of ordinary skill in the art to which this disclosure belongs. Patent applications, patents, and publications referred to herein to assist in the understanding of the aspects described are herewith incorporated by reference in their entirety.

In understanding the scope of the present application, the articles “a”, “an”, “the”, and “said” are intended to mean that there are one or more of the elements. Additionally, the term “comprising” and its derivatives, as used herein, are intended to be open-ended terms that specify the presence of the stated features, elements, components, groups, integers, and/or steps, but do not exclude the presence of other unstated features, elements, components, groups, integers and/or steps. The foregoing also applies to words having similar meanings such as the terms “including”, “having” and their derivatives.

It will be understood that any aspects describing as “comprising” certain components may also “consist of” or “consist essentially of”, wherein “consisting of” has a closed-ended or restrictive meaning and “consisting essentially of” means including the components specified but excluding other components except for materials present as impurities, unavoidable materials present as a result of processes used to provide the components, and components added for a purpose other than achieving the technical effect of the invention.

All ranges given herein include the end of the ranges and also any intermediate range points, whether explicitly stated or not.

Terms of degree such as “substantially”, “about” and “approximately” as used herein mean a reasonable amount of deviation of the modified term such that the end result is not significantly changed. These terms of degree should be construed as including a deviation of at least +/−5% of the modified term if the deviation would not negate the meaning of the word it modifies.

Although methods and materials similar or equivalent to those described herein can be used in the practice or testing of this disclosure, suitable methods and materials are described below. The abbreviation “e.g.” is used herein to indicate a non-limiting example. The word “or” is intended to include “and” unless the context clearly indicates otherwise.

We have found that administration of psilocin and/or psilocybin, or analogs thereof, both in “recreational” and in microdose amounts, results in an overall weight loss in individuals. Regular, recreational or microdose administration of Psilocin/psilocybin (or analogs thereof) can reduce food cravings, counteract compulsive overeating, and may even aid in improving quality of diet, by altering food choices. In fact, even a single dose has been found to be useful to this effect, though habitual use increases effectiveness. Recreational or microdose administration of psilocin/psilocybin (or analogs thereof) appear to have the additional weight loss effect of increasing metabolism, as well as decreasing food cravings or compulsive overeating, as well as actually altering food choices to less calorie dense foods. Each of these effects, especially when combined, can result in substantial and beneficial weight loss.

It is generally understood that obesity and overweightness is well correlated to an increased probability of diabetes and pre-diabetes conditions, and that these conditions can be ameliorated or sometimes even prevented through weight loss. Weight loss, and a decrease in compulsive overeating, can also aid in regulation of blood glucose in overweight/obese individuals with diabetes. Accordingly, recreational or microdose administration of psilocin/psilocybin (or analogs thereof), through its effect of weight loss in overweight individuals, may be useful in treatment or regulation of diabetes, and regulation of blood glucose, particularly in overweight or obese individuals. A recreational or microdose administration of psilocin/psilocybin (or analogs thereof) (and its resultant weight loss effect) may also reduce susceptibility to cardiovascular disease, high blood pressure, diabetes mellitus, hypertension, multiple sclerosis, erectile dysfunction, urinary incontinence, chronic renal failure, sleep apnea, asthma, and many forms of cancer, in overweight or obese individuals. Psilocin/psilocybin/analogs may also be used by non-overweight individuals, either to maintain a healthy weight, or to aid with compulsive eating disorder.

Either psilocin or psilocybin may be used, either alone or in combination. Analogs of psilocin or psilocybin, such as 4-SH-dimethyltryptamine, 1-methylpsilocin, 4-fluoro-N,N-dimethyltryptamine, O-acetylpsilocin, 4-hydroxy-N-methyl-N-isopropyltryptamine, and 4-hydroxy-N-methyl-N-ethyltryptamine, may also be used to similar effect, either alone or in combination with psilocin and/or psilocybin.

Dosing regimens can vary widely. For example, 10 mg to 5 g, preferably 50 mg to 500 mg, of dried mushroom, or approx. 60 micrograms to 35 mg, preferably 0.30 to 3 mg of pure psilocin may be taken per dose, with one or multiple doses taken per day. A typical dosage regime may be 100 to 400 mg of dried mushroom (equivalent to approximately 0.6 to 2.5 mg of psilocin) per day, in a single oral dose, taken 2 days in every 3. Another typical dosage regime may be, for example, 1 mg psilocin or psilocybin per day, taken in a single oral dose, taken 5 days of every 7. Dosing may be for a specified length of time (for example, for two months) or may be for an indefinite duration. Dosing may also be “as needed” or as desired by the user.

Without being limited by theory, it is believed that the beneficial diet and obesity treatment effects of psilocybin may be due to their 5-HT1A and 5-HT2C receptor agonist activity.

The above disclosure generally describes the present invention. A more complete understanding can be obtained by reference to the following specific examples. These examples are provided for purposes of illustration only and are not intended to be limiting unless otherwise specified. Thus the invention should in no way be construed as being limited to the following examples, but rather, should be construed to encompass any and all variations which become evident as a result of the teaching provided herein.

The following examples do not include detailed descriptions of conventional methods, which are well known to those or ordinary skill in the art and are described in numerous publications.

Without further description, it is believed that one of ordinary skill in the art can, using the preceding description and the following illustrative examples, make and utilize the compounds of the present invention and practice the claimed uses and methods. The following working examples therefore, specifically point out the typical aspects of the present invention and are not to be construed as limiting in any way the remainder of the disclosure.

### EXAMPLE 1

### Effects on Compulsive Eating Disorder

Individuals diagnosed with compulsive eating disorder are asked to self-report instances of compulsive eating over a two month period. They are then put on one of four treatment protocols over a two month period, and asked to self-report instances of compulsive eating: A) placebo treatment with 100 mg of sucrose in a gel tab, once a day with breakfast; B) 100 mg gel tab of Psilocybin cubensis fruiting bodies, once a day with breakfast (TruMood Activate microdosed organic psilocybin mushrooms obtained from Microdosenature.com); C) one square of psilocybin hazelnut chocolate (250 mg of psilocybin cubensis mushrooms from fruiting bodies per square, obtained from microdosenature.com), per day, with breakfast; D) A gel tab containing 3 mg of pure, synthetically produced, psilocybin in a pharmaceutically acceptable carrier. Individuals in groups B, C and D report a decrease in compulsive eating as compared to the first two months where they receive no treatment. Individuals in group A report generally similar amounts of compulsive eating as compared to the first two months.

### EXAMPLE 2

### Weight Loss

12 males all weighing between 250 and 275 lbs and all having BMI indexes between 30 and 35 kg/m² are split into two groups. The first is provided with placebo gel tabs; the second with indistinguishable gel tabs containing 2.5 mg of purified synthetically produced psilocin in a pharmaceutically acceptable carrier. Both groups are instructed to take one gel tab every morning, before breakfast, on weekdays, with weekends off, for 2 months. At the end of the two months, the first (placebo) group is found to have a similar weight as before the treatment, whereas the treatment group loses an average of 12 lbs each. The treatment group exhibits lower fasting glucose levels, decreased insulin resistance, lower lipid count in the blood stream, and lower levels of hypertension.

### EXAMPLE 3

### Sleep Apnea

12 females all suffering from sleep apnea, weighing between 225 and 250 lbs, and having BMI indexes above 30 kg/m² are split into two groups. The first is provided with a Hershey™'s chocolate bar, the second with psilocybin mushroom chocolates (microdosenature.com) containing 250 mg of organic psilocybin cubensis mushrooms from fruiting bodies per square. Both groups are instructed to take one square of chocolate every evening before going to sleep, every second night, for two months. All subjects are given a nocturnal polysomnography test before, and after, the two months of treatment. Females in the second group present with a decreased diagnosis of sleep apnea after treatment, as compared to before treatment. Females in the placebo group present with similar amounts of sleep apnea before and after treatment.

### EXAMPLE 4

### Food Selection

12 females are split into two groups. The first is provided with placebo gel caps, the second with psilocybin gel caps containing 250 mg of organic psilocybin cubensis mushrooms from fruiting bodies. Both groups are instructed to take one gel cap every morning. Subjects are asked to log their food intake for two months before, then two months during, treatment. Females in the second group record less high fat and carbohydrate-rich food intake, as compared to before treatment. Females in the placebo group do not have altered food intake.

### EXAMPLE 5

### Pre-Clinical Study of Weight-Loss Properties of Psilocybin with a High Calorie Diet

In order to determine whether psilocybin has anorectic—and therefore weight loss—properties, a preclinical study of the drug was conducted, using a well-validated animal model. Rodent models of food consumption and weight gain have a strong homology with humans, as rodents are omnivores with a very similar metabolic physiology. Prior art rodent studies that have tested drugs with known weight-loss benefits in humans have demonstrated similar effects in the animals (Hansen H H et al., Eur J. Pharmacol. 2010; 636(1-3):88-95; Vickers S P et al., Br J Pharmacol 2011; 164(4):1248-62). The goal of the present study was to cause accelerated weight gain with a high calorie diet, consisting of a highly palatable food with a large proportion of fat and carbohydrate, compared to standard animal food, which is designed to maintain a healthy weight. We evaluated whether treatment with two different doses of psilocybin could reduce the accelerated weight gain caused by this diet.

Adult, male Sprague-Dawley rats were purchased from the animal supplier, and then housed in pairs in polycarbonate cages, with access to water and standard rat food ad libitum. Rats were given a period of time to acclimate to the animal colony. They were then all given unlimited access to a highly palatable diet that is very high in carbohydrate and fat (consisting predominantly of a hazelnut-flavored chocolate spread combined with peanut butter) that has been used routinely in multiple published studies with rats to study rapid weight gain (Hansen et al., Acta pharmacologica Sinica. 2012; 33(2):194-200). Animals were randomly assigned to one of three treatment groups, which consisted of once daily administration of either a lower dose (5 mg/kg; n=7 animals) of psilocybin, a higher dose (20 mg/kg; n=7 animals) of psilocybin, or no psilocybin (n=12 animals). The psilocybin was supplied by the Katz Group at the University of Alberta, and was legally approved by an exemption from Health Canada.

Rats were administered the drug approximately two hours before the lights went off in the animal colony, which is when feeding typically commences. Animals were free to consume as much of the regular rat food or high calorie diet as they wished. They were weighed each day prior to drug treatment, to determine how much weight had been gained in the previous 24 hours. The study administered the drug over five consecutive days. Data were statistically analyzed by (SPSS Version 24 software package) using a one-way Analysis of Variance (ANOVA) with a LSD-post hoc test to detect between-group differences.

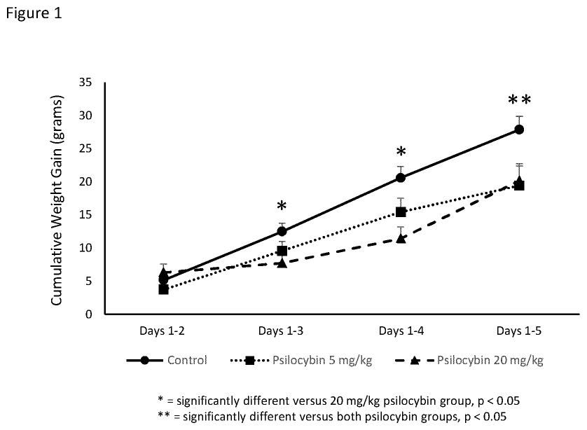

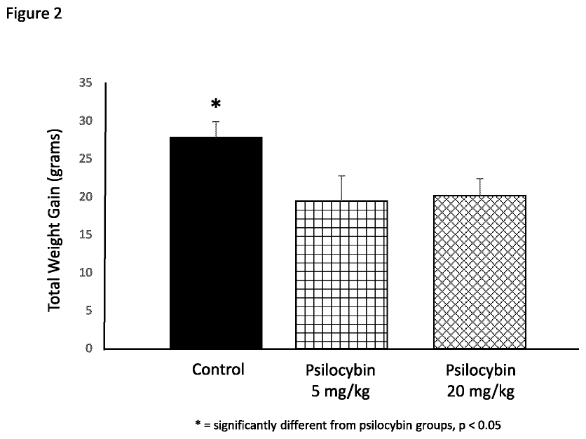

Animals quickly learned to eat the high calorie diet food. The control group, which did not receive treatment with psilocybin, exhibited a large and constant increase in body weight, of 5-8 grams per day (FIG. 1). In contrast, both of the psilocybin-treated groups showed a reduced amount of weight gain, between 1-8 grams per day. When the total weight gain was measured over the 5 day period (FIG. 2), the control group on average gained 27.8 grams, while the 5 mg/kg psilocybin group gained on average only 19.4 grams and the 20 mg/kg gained on average only 20.1 grams. This represents a 31% and 28% reduction in weight gain with psilocybin treatment. The formal analysis indicated that these effects were statistically significant. The ANOVA indicated that the two psilocybin treated groups showed less total weight gain than controls [F(2.25)=3.99, p<0.05]. When the data were analyzed on a day-by-day basis, comparing the cumulative weight gain for each day, the 20 mg/kg dose of psilocybin demonstrated a significantly reduced weight gain compared to controls by Day 3. By Day 5, both psilocybin-treated groups exhibited significantly decreased weight gain compared to controls.

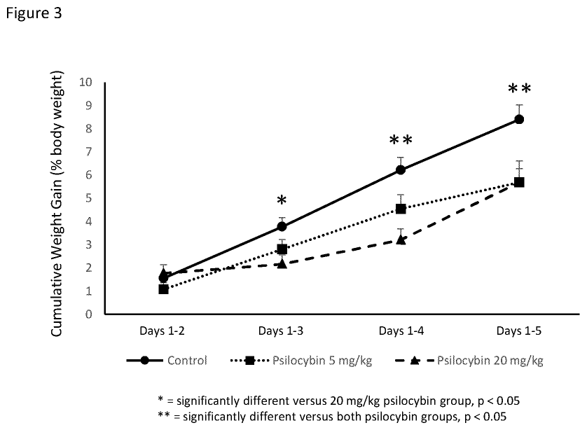

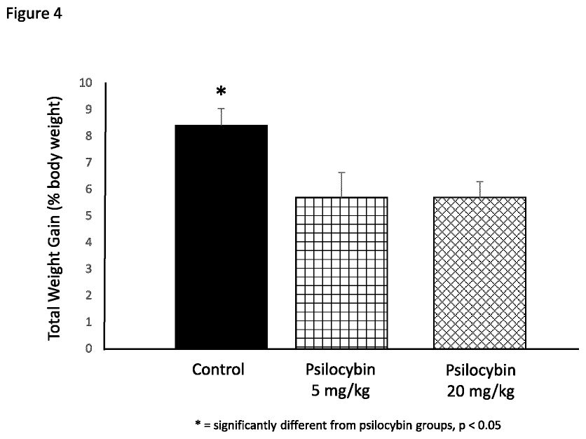

As the control group of rats were slightly lighter than the other groups on the first day of treatment (which would underestimate the effects of drug treatment, because larger rats gain more weight daily, in absolute terms), we also analyzed the weight gain relative to their baseline weight. We determined their daily weight gain in proportion to their weight prior to drug treatment. Results were expectedly similar to measures of absolute weight gain, but statistically more significant. A similar pattern of cumulative weight gain over the 5 days was evident (FIG. 3). Total relative weight gain was 8.4% in controls, and 5.7% in both of the psilocybin groups (FIG. 4)—a 32% decrease in relative weight gain in the latter two groups, which was statistically significant [F(2.25)=5.24, p=0.01]. On a day-by-day basis, the 20 mg/kg psilocybin group gained statistically less relative weight by day 3 compared to controls, and both psilocybin groups gained less relative weight by day 4 (p<0.05).

Controls rats readily consumed the high calorie, palatable diet and therefore gained weight quickly. This was significantly reduced by both doses of psilocybin, whereby weight gain was strongly decreased, by approximately one third. The effect of psilocybin was rapid, as it happened within days. The observation can only represent an effect on food consumption, as the animals had no opportunity to exercise in their cage, and so calorie expenditure was unaltered. These overall findings indicate that in a well-validated preclinical model, psilocybin is strongly able to reduce consumption of “junk” foods and maintain body weight at healthier levels. As these rodent models are highly predictive of similar effects in humans, clinical trials in overweight individuals are expected to yield similar results.

The above disclosure generally describes the present invention. Although specific terms have been used herein, such terms are intended in a descriptive sense and not for purposes of limitation.

All publications, patents and patent applications cited above are herein incorporated by reference in their entirety to the same extent as if each individual publication, patent or patent application was specifically and individually indicated to be incorporated by reference in its entirety.

Although preferable embodiments of the invention have been described herein in detail, it will be understood to those skilled in the art that variations may be made thereto without departing from the spirit of the invention or the scope of the appended claims.

1. A method of treating compulsive eating disorder, or of aiding in or causing weight loss, or of treating obesity, or of treating a complication associated with obesity, or of altering a diet, in an individual in need thereof, comprising administering an effective amount of a compound comprising psilocin, psilocybin, or an analog thereof.

2. (canceled)

3. (canceled)

4. (canceled)

5. A method of reducing food cravings, improving quality of diet, increasing metabolism, decreasing food intake in an individual in need or desire thereof, or aiding a healthy individual to maintain a healthy weight, comprising administering an effective amount of a compound comprising psilocin, psilocybin, or an analog thereof.

6. (canceled)

7. The method of claim 1 or 5 wherein the compound is contained within a dried mushroom powder of the genus Psilocybe.

8. The method of claim 1 or 5 wherein the compound is a purified psilocin or psilocybin in a pharmaceutically acceptable carrier.

9. The method of claim 8 wherein the purified psilocin or psilocybin is manufactured synthetically.

10. The method of claim 1 or 5 wherein the effective amount is 0.6 to 2.5 mg of psilocin, psilocybin, or analog thereof, per day.

11. The method of claim 7 wherein the effective amount is 50 to 500 mg of dried mushroom powder per day.

12. The method of claim 1 or 5 wherein the complication associated with obesity is selected from the group consisting of sleep apnea, abnormal regulation of blood glucose, diabetes mellitus, cardiovascular disease, high blood pressure, hypertension, multiple sclerosis, erectile dysfunction, urinary incontinence, chronic renal failure, asthma, and cancer.

13. The method of claim 1 or 5 wherein the administering is an oral administration.

14. The method of claim 1 or 5 wherein the analog thereof is 4-SH-dimethyltryptamine, 1-methylpsilocin, 4-fluoro-N,N-dimethyltryptamine, O-acetylpsilocin, 4-hydroxy-N-methyl-N-isopropyltryptamine, or 4-hydroxy-N-methyl-N-ethyltryptamine.

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)
